(19) United States
(12) Patent Application Publication
(10) Pub. No.: US 2026/0102156 A1
(43) Pub. Date: Apr. 16, 2026

(54) SOFT SUTURE CONSTRUCT WITH TUBULAR SHEATH

(71) Applicant: Arthrex, Inc., Naples, FL (US)

(72) Inventors: Julia CUNY, Naples, FL (US); Trent BOYER, Bonita Springs, FL (US); Andrew OSIKA, Naples, FL (US); Joshua J. BEST, Naples, FL (US)

(21) Appl. No.: 19/354,541

(22) Filed: Oct. 9, 2025

Related U.S. Application Data
(60) Provisional application No. 63/706,508, filed on Oct. 11, 2024.

(51) Int. Cl. A61B 17/06; A61B 17/04

(52) U.S. Cl. CPC A61B 17/06166; A61B 2017/0445

(57) ABSTRACT

A suture construct may include a tubular sheath, a repair strand, a retraction strand, and an activation strand. The tubular sheath may have a first end portion with a first opening, a second end portion with a second opening, and a lumen extending between the first opening and the second opening. The repair strand may be in the lumen and have a length extending through the first end portion, wherein the repair strand is configured to engage a soft tissue. The retraction strand may be configured to retract the tubular sheath. The activation strand may be in the lumen and configured to compress and anchor the tubular sheath in a bone tunnel. The activation strand may extend through the first opening and the second opening to tether the first end portion and the second end portion.

### PRIORITY

This application claims the benefit of priority to U.S. Provisional Application No. 63/706,508, filed Oct. 11, 2024, the entire disclosure of which is incorporated herein.

### TECHNICAL FIELD

This disclosure relates generally to a soft suture construct, and more specifically, directed to a suture construct having a tubular sheath.

### BACKGROUND

When soft tissue, such as a ligament or a tendon, tears or becomes detached from a bone, surgery is usually required to reattach or reconstruct the tissue. Techniques and devices have been developed generally involve securing an anchor in a hole provided in the bone tissue and tying the soft tissue with suture to an anchor. The bone anchor can be a hard anchor or a soft anchor.

### SUMMARY

The present inventors recognize that hard suture anchors often require substantial bone removal for securing the anchor due to the bulky nature of the hard suture anchors. Hard anchors also typically require rotating the bone anchor to be secured in place with a threaded engagement, which can be difficult to achieve in minimally invasive procedures. Furthermore, soft anchors can involve complex braiding patterns that can be difficult to manufacture.

Thus, a first aspect of the present disclosure is directed to a suture construct comprising: a tubular sheath having a first end portion with a first opening, a second end portion with a second opening, and a lumen extending between the first opening and the second opening; a repair strand in the lumen and having a length extending through the first end portion, wherein the repair strand is configured to engage a soft tissue; a retraction strand configured to retract the tubular sheath; and an activation strand in the lumen and configured to compress and anchor the tubular sheath in a bone tunnel, wherein the activation strand extends through the first opening and the second opening to tether the first end portion and the second end portion.

In some embodiments, the suture construct may have one or more of the following features. The activation strand may be a closed loop. The retraction strand may include a loop extending around and/or through the tubular sheath. The repair strand may have a second length extending through the second end portion. The repair strand may be fixed to a portion of the tubular sheath. The repair strand may be fixed at a central portion between the first opening and the second opening. The repair strand may be spliced through the tubular sheath. The repair strand may be spliced at a first transverse opening and a second transverse opening of the tubular sheath. The first transverse opening and the second transverse opening may be spaced apart along a longitudinal axis the tubular sheath. The repair strand may form a loop extending through the first transverse opening and the second transverse opening. The first transverse opening and the second transverse opening may be spaced apart transverse to a longitudinal axis of the tubular sheath. The repair strand may be looped at least partially around the tubular sheath. The repair strand may form a knot inside or outside of the tubular sheath. The repair strand forms the knot inside of the tubular sheath. The repair strand may be fixed to the tubular sheath with one or more stitches. The repair strand may include a loop, the repair strand extends at least partially around the tubular sheath and through the loop, and the repair strand is spliced into the tubular sheath. The repair strand may have an opening, and the tubular sheath is passed through the opening. The suture construct may include a shuttle associated with the repair strand. The shuttle may be spliced through a portion of the repair strand. The portion of the repair strand may be disposed entirely within the tubular sheath. The portion of the repair strand may be at least partially outside of the tubular sheath.

A second aspect of the present disclosure is directed to a method of forming a suture construct from a tubular sheath having a lumen, a first end portion, and a second end portion, the method comprising: inserting a repair strand into the lumen of the tubular sheath, such that a length of the repair strand extends through the first end portion; extending the repair strand through a first transverse opening and a second transverse opening of the tubular sheath; inserting an activation strand into the tubular sheath; and securing a retraction strand to the tubular sheath.

In some embodiments, the method may have one or more of the following features. The activation strand may extend through a first opening of the first end portion and a second opening of the second end portion to tether the first end portion and the second end portion. The first transverse opening and the second transverse opening may be spaced apart along a longitudinal axis of the tubular sheath. The first transverse opening and the second transverse opening may be spaced apart transverse to a longitudinal axis of the tubular sheath. The method may further include looping the repair strand around the tubular sheath. The method may further include forming a knot with the repair strand inside or outside of the tubular sheath.

### BRIEF DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific methods, devices, and systems disclosed. In the drawings:

FIG. 1 illustrates a first embodiment of a suture construct according to the present disclosure.

FIG. 2 illustrates a cross-sectional view of the suture construct of FIG. 1.

FIG. 3 illustrates a second embodiment of the suture construct according to the present disclosure.

FIG. 4 illustrates a cross-sectional view of the suture construct of FIG. 3.

FIG. 5 illustrates an embodiment of a shuttle of the suture construct of FIGS. 3 and 4.

FIGS. 6A-C illustrate an exemplary method of tissue repair using an embodiment of the suture construct of FIGS. 1-4 according to the present disclosure.

FIGS. 7A-C illustrates a first exemplary method of loading the suture construct of FIGS. 1-4 according to the present disclosure.

FIGS. 8A-D illustrate a second exemplary method of loading the suture construct of FIGS. 1-4 according to the present disclosure.

FIGS. 9A-D illustrate a third exemplary method of loading the suture construct of FIGS. 1-4 according to the present disclosure.

FIGS. 10A-C illustrate a fourth exemplary method of loading the suture construct of FIGS. 1-4 according to the present disclosure.

FIG. 11 illustrates a fifth exemplary method of loading the suture construct of FIGS. 1-4 according to the present disclosure.

FIGS. 12A-C illustrate a sixth exemplary method of loading the suture construct of FIGS. 1-4 according to the present disclosure.

FIGS. 13A-C illustrate a seventh exemplary method of loading the suture construct of FIGS. 1-4 according to the present disclosure.

FIGS. 14A-B illustrate an eighth exemplary method of loading the suture construct of FIGS. 1-4 according to the present disclosure.

FIGS. 15A-C illustrate a ninth exemplary method of loading the suture construct of FIGS. 1-4 according to the present disclosure.

Aspects of the disclosure will now be described in detail with reference to the drawings, wherein like reference numbers refer to like elements throughout, unless specified otherwise.

### DETAILED DESCRIPTION OF ILLUSTRATIVE EMBODIMENTS

The present disclosure relates to a suture construct, methods of using the suture construct for repairing tissue, and methods of making the suture construct. The suture construct of the present disclosure may be an all-suture implant. The suture construct may be designed to facilitate anchoring and securing a soft tissue to bone, for example through a portal in a minimally invasive procedure. The suture construct may be double-loaded allowing for two repair suture ends in a single anchor pass. In some embodiments, the suture construct may facilitate inline repairs in a knotless technique with one or more pre-loaded shuttles. The all-suture design of the suture construct may allow minimal bone removal with a smaller drill pin. The design of the suture construct may further allow for a simplified method of repairing tissue as compared to conventional repair techniques. For example, a simplified inserter may be used to the suture construct for being pulled instead of pushed into place. In an exemplary application, the suture construct may be used to repair a meniscal root repair by anchoring the same in a transtibial tunnel. The suture construct may eliminate the need for posterior knee portals and may allow for direct visualization of the repair site. Furthermore, the suture constructs of the present disclosure may be easily manufactured with a tubular sheath.

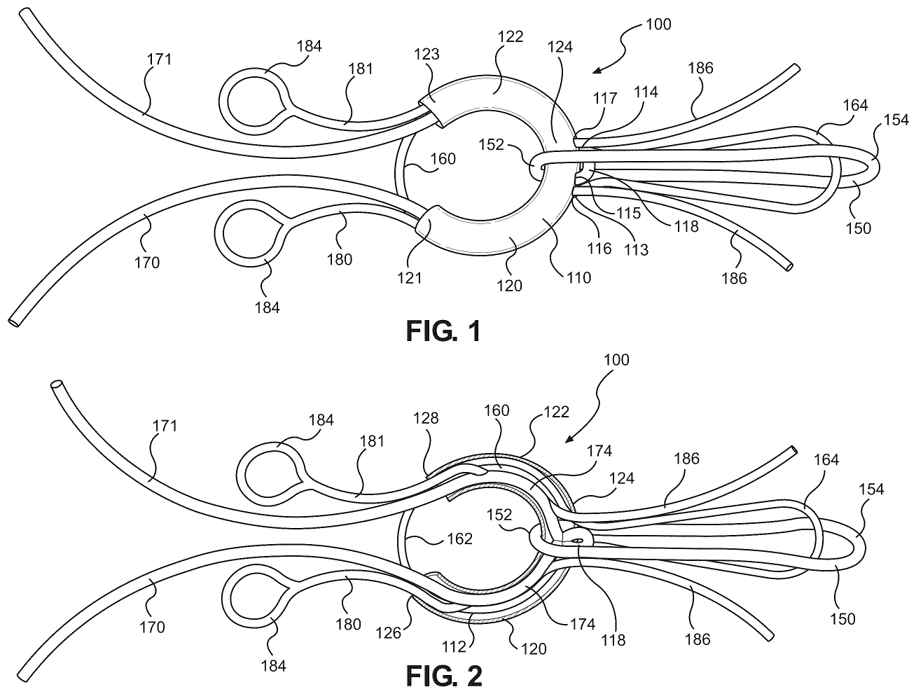

FIGS. 1 and 2 illustrate a first embodiment of a suture construct 100. The suture construct 100 may include a tubular sheath 110, a retraction strand 150, an activation strand 160, at least one repair strand 170, 171, and at least one shuttle 180, 181. The tubular sheath 110 may have a lumen 112 that receives the activation strand 160, the at least one repair strand 170, 171, and the at least one shuttle 180, 181. The at least one repair strand 170, 171 and at least one shuttle 180, 181 may extend from the tubular sheath 110 in a first direction to be manipulated by a user. The retraction strand 150 and an activation strand 160 may extend from the tubular sheath 10 in a second direction opposite of the first direction to be manipulated by the user. The retraction strand 150 may be pulled to pull the tubular sheath 110 into a bone tunnel. The activation strand 160 may be pulled to compress the tubular sheath 110 to anchor the suture construct 100 in the bone tunnel. The at least one repair strand 170, 171 may be configured to engage a soft tissue to secure the soft tissue to the bone. In some embodiments, the one or more shuttles 180, 181 may be provided to thread the at least one repair strand 170, 171 to extend or be spliced into itself and provide a knotless mechanism to secure the soft tissue. In some embodiments, the one or more shuttles 180, 181 may be omitted.

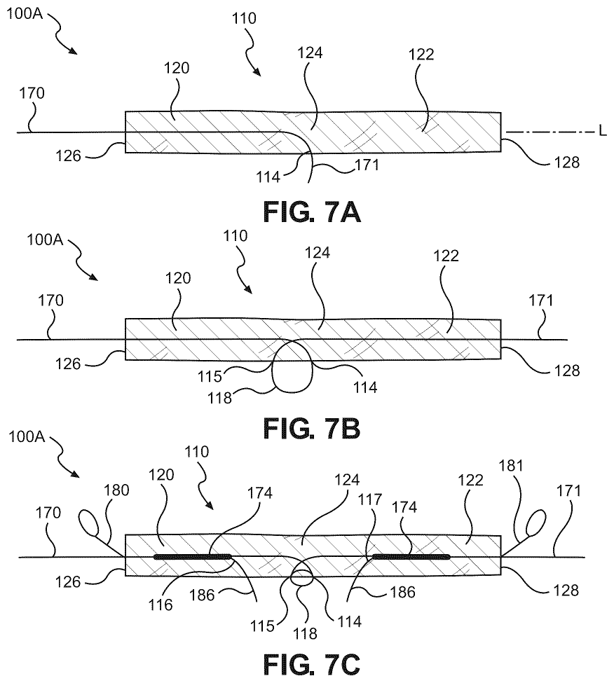

The tubular sheath 110 may be formed by a tubular wall defining the lumen 112. The tubular sheath 110 may have a first end portion 120, a second end portion 122, and a central portion 124 between the first end portion 120 and the second end portion 122. The activation strand 160 may shape the tubular sheath 110 such that the first end portion 120 and the second end portion 122 are spaced apart or disconnected distal of the central portion 124, as illustrated in FIGS. 1 and 2. For example, the activation strand 160 may shape the tubular sheath 110 into a substantially C-shaped configuration such that a first terminal end 121 of the first end portion 120 may be spaced apart or disconnected from a second terminal end 123 of the second end portion 122, as illustrated. The first end portion 120 may have a first opening 126 at a first end of the lumen 112, and the second end portion 122 may have a second opening 128 at a second end of the lumen 112. The first opening 126 may be through the first terminal end 121 of the first end portion 120 or may be transverse and spaced from the first terminal end 121. Similarly, the second opening 128 may be through the second terminal end 123 of the second end portion 122 or may be transverse and spaced from the second terminal end 123. The first opening 126 and the second opening 128 may be along a longitudinal axis L of the tubular sheath 110, as illustrated in the straightened configuration of FIGS. 7-15. The tubular wall of the tubular sheath 110 may extend continuously between the first terminal end 121 and the second terminal end 123. The tubular sheath 110 may have a single non-bifurcated lumen 112 extending along the longitudinal axis L.

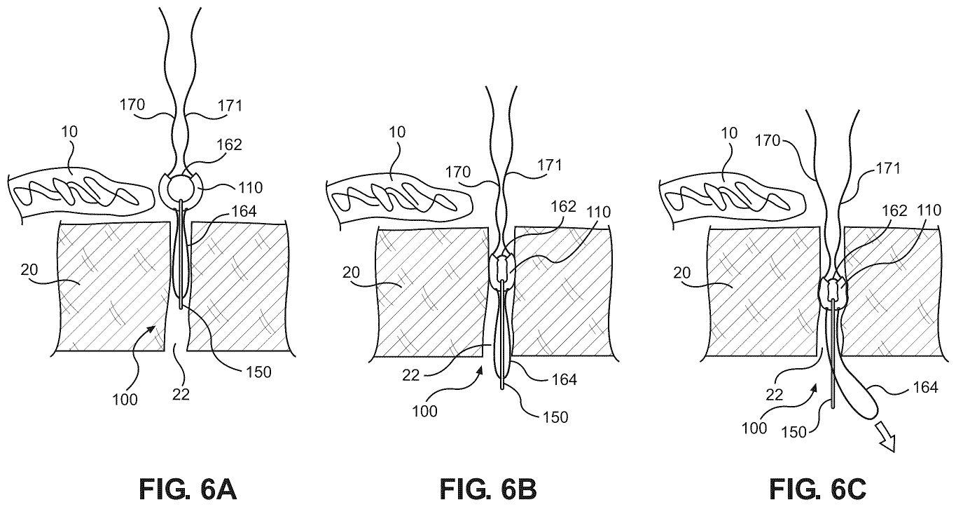

The tubular sheath 110 may be formed of a flexible material such that the first end portion 120 and the second end portion 122 may be longitudinally compressed or bunched. The first end portion 120 and the second end portion 122 may be compressed or bunched between an uncompressed configuration (e.g., FIGS. 6A, B) and a longitudinally compressed configuration (e.g., FIG. 6C) in order to set or anchor the tubular sheath 110 inside of the bone tunnel. In some embodiments, the tubular sheath 110 may be elastic. The tubular sheath 110 may be formed of a plurality of strands, such as yarns, fibers, and/or filaments that are woven, braided, and/or knitted into the tubular configuration. The tubular sheath 110 may be made of at least one ultrahigh molecular weight polyethylene (UHMWPE) strand, at least one polyester strand, and/or at least one elastic strand. For example, in some embodiments, the tubular sheath 110 may be constructed of a plurality of UHMWPE strands, for example, entirely of UHMWPE strands. In some embodiments, the tubular sheath 110 may be constructed of a plurality of polyester strands, for example, entirely of polyester strands. In some embodiments, the tubular sheath 110 may be constructed of a plurality of elastic strands, for example, entirely of elastic strands. In some embodiments, the tubular sheath 110 may be constructed of at least one UHMWPE strand and at least one polyester strand. In some embodiments, the tubular sheath 110 may be constructed of at least one UHMWPE strand and at least one elastic strand. In some embodiments, the tubular sheath 110 may be constructed of at least one polyester and at least one elastic strand. In some embodiments, the tubular sheath 110 may be constructed of at least one UHMWPE strand, at least one polyester strand, and at least one elastic strand. The elastic strand may be composed of elastane. The at least elastic strand may provide elasticity, while the at least one UHMWPE strand and/or the at least one polyester stand may add strength and limit the elongation of the tubular sheath 110. The tubular sheath 110 may be loosely braided to facilitate splicing of the activation strand 160, the at least one repair strand 170, 171, and/or the at least one shuttle 180, 181. The loose braiding of the tubular sheath 110 may, additionally or alternatively, facilitate longitudinal compression of the end portions 120, 122.

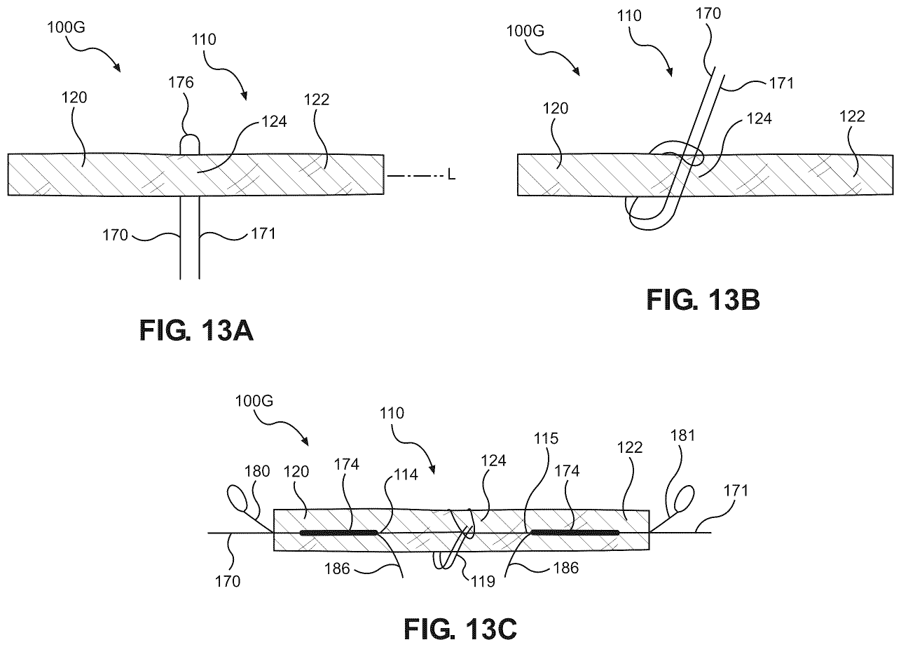

The retraction strand 150 may be configured to retract the tubular sheath 110. The retraction strand 150 may extend at least partially through and/or around the tubular sheath 110. As illustrated in FIGS. 1 and 2, the retraction strand 150 may have a distal portion 152 extending around a distal circumference of the central portion 124 of the tubular sheath. In some embodiments, the distal portion 152 may be fixed to the central portion 124 with an adhesive and/or weld. Additionally or alternatively, the distal portion 152 may extend or be spliced into the central portion 124 at one or more locations around the distal circumference of the wall. A proximal portion 154 of the retraction strand 150 may be configured to be manipulated by the user. The central positioning of the retraction strand 150 may allow retraction of the tubular sheath 110 such that the end portions 120, 122 may engage the sides of the bone tunnel. As illustrated in FIGS. 1 and 2, the retraction strand 150 may be a closed loop forming the distal portion 152 and the proximal portion 154. In some embodiments (not shown), the retraction strand 150 may have a fixed distal loop and a proximal tail where the fixed distal loop extends around the tubular sheath 110, similar to that illustrated for the shuttles 180, 181. In some embodiments (not shown), the retraction strand 150 may be a strand having slip knot at its distal end around the tubular sheath 110. In some embodiments (not shown), the retraction strand 150 may be a strand forming a luggage tag around the tubular sheath 110, similar to that as illustrated in FIG. 13A-C. In some embodiments (not shown), the retraction strand 150 may be a free strand passed through and/or around the tubular sheath and not forming a closed loop and/or knot.

The activation strand 160 may be a flexible strand that extends through the tubular sheath 110. Returning to FIGS. 1 and 2, the activation strand 160 may extend or be spliced into the tubular sheath 110 at one or more locations, with a distal portion 162 looped through the lumen 112 of the tubular sheath 110 and a proximal portion 164 extending out of the tubular sheath 110 to be manipulated by the user to compress the tubular sheath 110. A first length of the distal portion 162 may extend through the lumen 112 of the first end portion 120. A second length of the distal portion 162 may extend through the lumen 112 of the second end portion 122. The distal portion 162 may extend through the first opening 126 of the first end portion 120 and the second opening 128 of the second end portion 122. The distal portion 162 may extend across a space between the first terminal end 121 and the second terminal end 123 to form a loop that physically connects or tethers the first end portion 120 and the second end portion 122. FIGS. 1 and 2 illustrate the activation strand 160 extending through the same openings 126, 128 as the at least one repair strand 170, 171 and the shuttles 180, 181. This configuration may simplify loading of the tubular sheath 110. However, it is also contemplated that the activation strand 160 may extend through different openings than the at least one of the at least one repair strand 170, 171 and/or the shuttles 180, 181. In some embodiments (not shown), the activation strand 160 may enter and exit one or more times along the length of at least one of the end portions 120, 122, at the same or different locations. For example, the activation strand 160 may extend or be spliced into and out of the tubular sheath 110 forming openings at different locations along the length of the wall of each of the end portions 120, 122 to more evenly distribute the bunching along its length.

The proximal portion 164 of the activation strand 160 may extend or be spliced proximally out of the tubular sheath 110 at one or more locations to be manipulated by the user. The proximal portion 164 may have a first length and a second length that extend out of the tubular sheath 110. The first length of the proximal portion 164 may extend or be spliced through a first transverse opening 113 at the central portion 124 and the second length of the proximal portion 164 may extend or be spliced through a second transverse opening (not shown) at the central portion 124. The first transverse opening 113 and the second transverse opening may be through the wall of the tubular sheath 110 transverse to the longitudinal axis L. The first transverse opening 113 and the second transverse opening may be spaced apart along the longitudinal axis L of the tubular sheath 110, as illustrated in FIGS. 7-15. Extending the first and second lengths of the proximal portion 164 out of different openings 113 may help prevent the proximal portion 164 from being pulled into the lumen 112 of the tubular sheath 110. However, it is also contemplated that the first and second lengths of the proximal portion 164 may extend out of the same opening. The activation strand 160 may be a loop. In some embodiments, the activation strand 160 may be a closed loop such that the first and second lengths of the proximal portion 164 are joined to form a closed proximal end. In some embodiments, the activation strand 160 may have free proximal ends.

The at least one repair strand 170, 171 may be received in the tubular sheath 110 and be configured to engage the soft tissue. The at least one repair strand 170, 171 may have free ends extending from at least one of the openings 126, 128 for use in the tissue repair procedure. As illustrated in FIGS. 1 and 2, the at least one repair strand may have a first length 170 received in the first end portion 120 and extend out of the first opening 126 and a second length 171 received in the second end portion 122 and extend out of the second opening 128. Each of the at least one repair strand 170, 171 may be configured to be passed through and/or around the soft tissue to secure the soft tissue to the bone through knotted or knotless fixtures. The at least one repair strand 170, 171 may be pre-loaded in the tubular sheath 110 during manufacturing and prior to insertion into the body.

In some embodiments, the first length 170 and the second length 171 may be formed from a single repair strand. The repair strand 170, 171 may be fixed longitudinally to the tubular sheath 110. Fixing the repair strand 170, 171 to the tubular sheath 110 may improve the ability to tension the repair strand 170, 171, such as while performing a bridging technique. In some embodiments, the repair strand 170, 171 may be fixed to the tubular sheath 110 by being spliced and/or looped through the wall of the tubular sheath 110. As illustrated in FIGS. 1 and 2, the repair strand 170, 171 may extend through or be spliced at a third transverse opening 114 and at a fourth transverse opening 115. The third transverse opening 114 and fourth transverse openings 115 may be through the wall of the tubular sheath transverse to the longitudinal axis L. In some embodiments, the third transverse opening 114 and fourth transverse openings 115 may be spaced apart along the longitudinal axis L. In some embodiments, the third transverse opening 114 and fourth transverse openings 115 may be spaced apart transverse to the longitudinal axis L. As further illustrated and discussed with reference to FIGS. 7A-C, the repair strand 170, 171 may form a loop 118 at least partially outside of the tubular sheath 110 and/or at least partially inside of the tubular sheath 110. Thus, the loop 118 may extend or be spliced through the tubular sheath 100, extending both outside of the tubular sheath 110 and inside of the tubular sheath 110. The loop 118 may extend through the third transverse opening 114 and the fourth transverse opening 115 of the central portion 124. The loop 118 may extend 360° or be a closed loop (when viewed laterally), such that the repair strand 170, 171 may extend through the lumen 112 past the fourth transverse opening 115, through the third transverse opening 114, bend backwards outside of the lumen 112, back into the lumen 112 through the fourth transverse opening 115, then through the lumen 112 past the third transverse opening 114.

Additionally or alternatively, the repair strand 170, 171 may be tied into a knot 173 inside or outside of the lumen 112. Additionally or alternatively, the repair strand 170, 171 may be fixed to the tubular sheath 110 by one or more stitches extending through the tubular sheath 110. Additionally or alternatively, the repair strand 170, 171 may be fixed to the tubular sheath 110 with an adhesive and/or a weld. Exemplary embodiments of the fixation of the at least one repair strand 170, 171 are further discussed with regard to FIGS. 7-15. In some embodiments (not illustrated), the first length 170 and the second length 171 may be formed of separate repair strands. Additionally or alternatively, the at least one repair strand 170, 171 may not be fixed to the tubular sheath 110 and slide within the lumen 112 of the tubular sheath 110 (not illustrated).

As further illustrated in FIGS. 1 and 2, the at least one shuttle 180, 181 may be provided and associated with the at least one repair strand 170, 171. The at least one shuttle 180, 181 may extend or be spliced through a portion 174 of the at least one repair strand 170, 171. The spliced configuration of the shuttle 180, 181 may allow the soft tissue to be fixed with a knotless self-locking mechanism and allow the user to control the tension of the repair strand 170, 171 by pulling a proximal portion 186 of the at least one shuttle 180, 181. The at least one shuttle 180, 181 may be preloaded into the tubular sheath 110 by being spliced into the at least one repair strand 170, 171 prior to inserting into the at least one repair strand 170, 171 into the tubular sheath 110, as illustrated in FIGS. 7-15. Each shuttle 180, 181 may have a loop 184 configured to receive a free end of one of the repair strands 170, 171. The loop 184 may be at a distal end of the shuttle 180, 181. As illustrated in FIGS. 1 and 2, the proximal portion 186 of the first shuttle 180 may extend through a fifth transverse opening 116, and a proximal portion 186 of the second shuttle 181 may extend through a sixth transverse opening 117, each to be accessed by the user. The fifth transverse opening 116 and the sixth transverse opening 117 may be spaced apart along the longitudinal axis L.

A first shuttle 180 may extend proximally from its distal end into the first opening 126 and into the lumen 112 of the first end portion 120. The first shuttle 180 may extend or be spliced into and out of a portion 174 of the first length of repair strand 170. In some embodiments (as illustrated in FIGS. 1 and 2), the entire spliced portion 174 may be inside of the lumen 112 of the tubular sheath 110. The first shuttle 180 may extend through the fifth transverse opening 116 of the tubular sheath 110 to be manipulated by the user. Thus, after passing a free end of the first length of repair strand 170 through the loop 184, the user may pull the proximal portion 186 of the first shuttle 180 to pull the free length of repair strand 170 into the first opening 126 and into the spliced portion 174 of the first repair strand 170 such that it doubles on itself inside of the tubular sheath 110. The spliced portion 174 may frictionally engage the free of the first length of repair strand 170 inside of the spliced portion 174 of the first length of repair strand 170 to form a cinched loop knotless construct. Continued pulling of the first shuttle 180 may pull the first shuttle 180 out of the spliced portion 174 of the first length of repair strand 170 and disengage the free end of the first repair strand 170 from the loop 184. The first shuttle 180 may then be pulled out of the fifth transverse opening 116 of the tubular sheath 110 for removal from the surgical site.

Similarly, a second shuttle 181 may extend proximally from its distal end into the second opening 128 and into the lumen 112 of the second end portion 122. The second shuttle 181 may extend or be spliced into and out of a portion 174 of the second length of repair strand 171. In some embodiments (as illustrated in FIGS. 1 and 2), the entire spliced portion 174 may be inside of the lumen 112 of the tubular sheath 110. The first shuttle 180 may extend through the sixth transverse opening 117 of the tubular sheath 110 to be manipulated by the user. Thus, after passing a free end of the second length of repair strand 171 through the loop 184, the user may pull the proximal portion 186 of the second shuttle 181 to pull the second length of repair strand 171 into the second opening 128 and into the spliced portion 174 of the second length of repair strand 171 such that it doubles on itself inside the tubular sheath 110. The spliced portion 174 may frictionally engage the free of the second length of repair strand 171 inside of the spliced portion 174 of the second length of repair strand 171 to form a cinched loop knotless construct. Continued pulling of the second shuttle 181 may pull the second shuttle 181 out of the spliced portion 174 of the second length of repair strand 171 and disengage the free end of the second length of repair strand 171 from the loop 184. The second shuttle 181 may then be pulled out of the sixth transverse opening 117 of the tubular sheath 110 for removal from the surgical site.

The at least one repair strands 170, 171 may be a high-strength suture, formed from at least one strand of UHMWPE that can easily be spliced. In some embodiments, the at least one repair strands 170, 171 may be a FiberWire® suture, produced by Arthrex and disclosed in U.S. Pat. No. 6,716,234, the entire disclosure of which is incorporated herein by reference. FiberWire® suture may be formed of UHMWPE braided with at least one other fiber to form lengths of suture material. The Fiber Wire® suture may include a core within a hollow braided construct, the core being a twisted yarn of UHMWPE. Additional non-limiting alternatives of the at least one repair strands 170, 171 include TigerWire® suture, FiberChain® suture, or TightRope® suture (all produced by Arthrex), although other cored or coreless sutures may be utilized. Additionally or alternatively, the at least one repair strands 170, 171 may be a flat suture tape, such as FiberTape® produced by Arthrex. In some embodiments, the suture construct 100 may include one or more secondary fixation devices, such as a button attached to the at least one repair strands 170, 171. The at least one shuttle 180, 181 may be a FiberLink™ suture, a TigerLink™ suture (both produced by Arthrex), or a Nitinol loop.

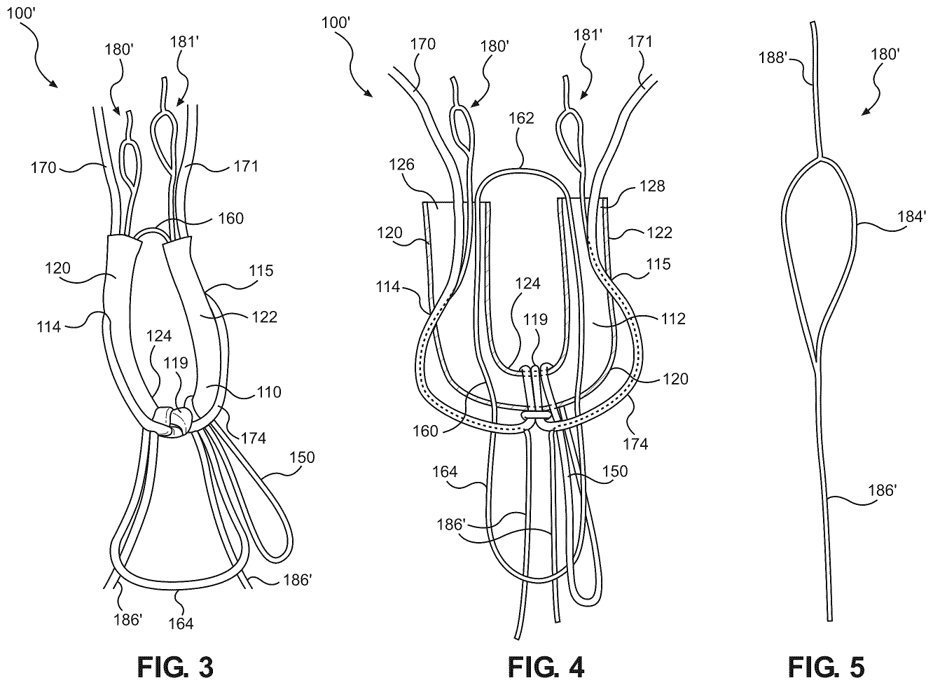

FIGS. 3 and 4 illustrates a second embodiment of the suture construct 100′. The suture construct 100′ may embody features of the first embodiment of the suture construct 100, the entire disclosure of which are incorporated herein by reference. For example, as further discussed with reference to the first embodiment of FIGS. 1 and 2, the suture construct 100′ may include the tubular sheath 110, the retraction strand 150, the activation strand 160, at least one repair strand 170, 171, and at least one shuttle 180′, 181′. The tubular sheath 110 may have a lumen 112 that receives the activation strand 160, the at least one repair strand 170, 171, and the at least one shuttle 180′, 181′. The retraction strand 150 may be pulled to pull the tubular sheath 110 into a bone tunnel. The activation strand 160 may be pulled to compress the tubular sheath 110 to anchor the suture construct 100 in the bone tunnel. The at least one repair strand 170, 171 may be configured to engage a soft tissue to secure the soft tissue to the bone. In some embodiments, the one or more shuttles 180′, 181′ may be provided to thread the at least one repair strand 170, 171 to extend or be spliced into itself and provide a knotless mechanism to secure the soft tissue. In some embodiments, the one or more shuttles 180′, 181′ may be omitted.

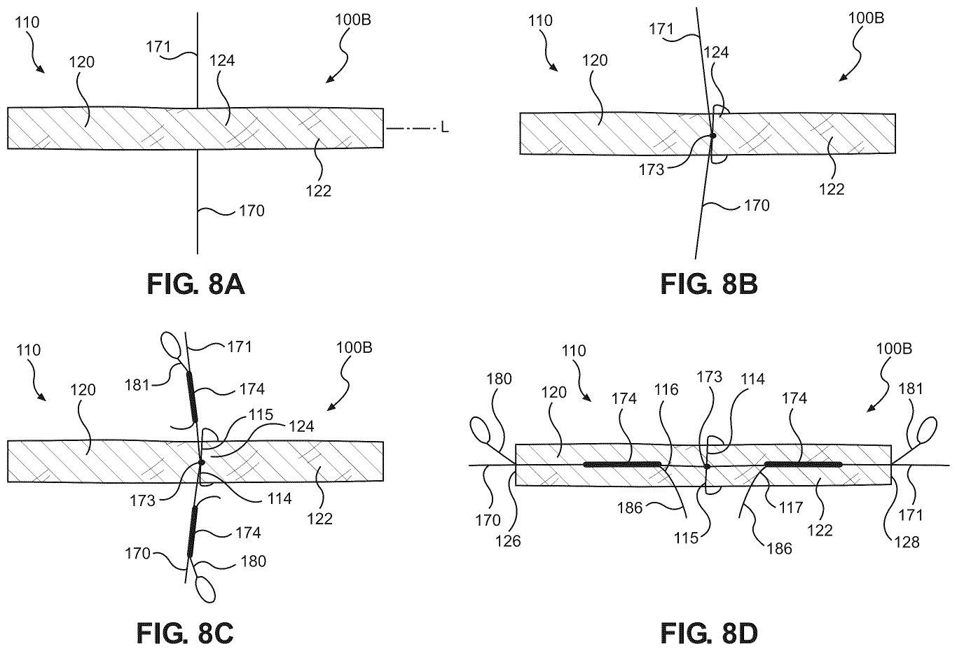

As further illustrated, the at least one repair strand 170, 171 may be a single strand secured to the tubular sheath 120 by forming at least one loop 119 at least partially around an outer surface of the tubular sheath 120. For example, in some embodiments, the one repair strand 170, 171 may be knotted inside or outside of the lumen 112, as discussed with reference to the embodiments of FIGS. 8A-C and 9A-C. In some embodiments, the repair strand 170, 171 may, additionally or alternatively, be secured to the tubular sheath 110 with a luggage tag attachment. The luggage tag attachment may be formed by looping the repair strand 170, 171 around the tubular sheath 120 and through itself, as illustrated with reference to FIGS. 13A-C. After forming the attachment with the loop 119, the first length 170 may extend or be spliced through a first transverse opening 114, into the lumen 120, and out of the first opening 126. Similarly, the first length 170 may extend or be spliced through a second transverse opening 115, into the lumen 120, and out of the second opening 128. The repair strand 170, 171 may be anchored to the central portion 124 of the tubular sheath 110 with the at least one loop 119. At least a portion of the spliced portion 174 may be positioned outside of the tubular sheath 110 and be spliced into each of the end portions 120, 122. Thus, the configuration of the repair strand 170, 171 may allow for precisely controlled displacement. The at least one shuttle 180′, 181′ may be spliced into the repair stand 170 at a first portion outside of the lumen 112 of the tubular sheath 110 and out of the repair strand 170 at a second portion as or after the repair strand 170, 171 enters into the lumen 112.

As further illustrated in FIGS. 3-5, a second embodiment of the at least one shuttle 180′, 181′ may have a proximal portion 186′, a loop 184′, and a distal portion 188′. Thus, the at least one shuttle 180′, 181′ may be provided with a double-tail configuration with two lengths of strand 186′, 188′ extending in opposite direction of the loop 184′. The distal portion 188′ may extend distally of the loop 184′ to be grasped and facilitate manipulation of the at least one shuttle 180′, 181′. Although FIG. 5 illustrates the shuttle 180′, it should be readily understood that the shuttle 181′ may have similar components. As discussed with reference to the first embodiment of FIGS. 1 and 2, the proximal portion 186′ may be spliced through the at least one repair strand 170, 171 to be pulled by the user to splice the at least one repair strand 170, 171 into itself to produce a knotless anchor construct. The first (180, 181) and second (180′, 181′) embodiments of the at least one shuttle may be interchangeably implemented in the first (100) and second (100′) embodiments of the suture construct.

FIGS. 6A-C illustrate an exemplary method of securing a soft tissue 10 to a bone tissue 20 by anchoring the suture construct 100 in a bone tunnel 22 through the bone tissue 20. Although illustrated and discussed with reference to the first embodiment of the suture construct 100, it should be readily understood that the method is applicable to the second embodiment of the suture construct 100′. The suture construct 100 may be used in a variety of techniques to attach the soft tissue 10 to the bone tissue 20. In one example, the soft tissue 10 may be a meniscal tissue and the bone tissue 20 may be a tibia, and the method may be implemented in a transtibial meniscal root repair. The bone tunnel 22 may be formed or drilled transversely through the bone tissue 20 by a drill pin of reduced size. The user may initially pass a suture threader (e.g., a nitinol wire, not shown) through a first access opening of the body (not shown), through the bone tunnel 22, and through a second access opening of the body (not shown). The first and second access openings may be anterior, such that no posterior knee ports would be required in the procedure. The first access opening may be defined by a pin drilled through a transtibial tunnel, where the pin may be cannulated to receive the suture threader. The second access opening may be defined by a button cannula at an exit of the bone tunnel 22.

The user may engage the suture construct 100 at the proximal portion 154 of the retraction strand 150 with the suture threader outside of the second port. The user may pull the suture threader from the first port. The user may thus pass the suture construct 100 through the second access opening in a retrograde fashion and into the bone tunnel 22. Thus, as illustrated in FIGS. 6A-B, the suture construct 100 may be passed into the bone tunnel 22 such that the retraction strand 150 and the central portion 124 are passed into the bone tunnel 22 before the end portions 120, 122. As illustrated in FIG. 6B, the suture construct 100 may be placed into the bone tunnel 22 such that the tubular sheath 110 is just below a surface of the bone tissue 20 (e.g., a tibial plateau) and the retraction strand 150 extends through the bone tunnel 22. The bone tunnel 22 may slightly compress the end portions 120, 122 laterally. The at least one repair strands 170, 171 and the at least one shuttle 180, 181 may extend from the bone tunnel 22 to be available for use in securing the soft tissue 10. The suture construct 100 may have a length such that the retraction strand 150 may extend from the bone tunnel 22 through the first access opening to be accessed by the user. Similarly, the distal ends of the at least one repair strands 170, 171 and the at least one shuttle 180, 181 may extend from the bone tunnel 22 through the second access opening to be accessed by the user. The at least one shuttle 180, 181 are not shown in FIGS. 6A-C for clarity purposes.

As illustrated in FIG. 6C, once the suture construct 100 is pulled into the body and positioned in the bone tunnel 22, the proximal portion 164 of the activation strand 160 may be pulled or tensioned to bunch or compress the tubular sheath 110. The activation strand 160 may be pulled in the retrograde direction to bunch the end portions 120, 122 and anchor the fixation device within the bone tunnel 22. Pulling the proximal portion 164 may pull the distal portion 162 of the activation strand 160 to shorten and laterally expand the end portions 120, 122. In the bunched configuration, the tubular sheath 110 may frictionally engage the inner surface of the bone tunnel 22 and/or create an interference fit to anchor the tubular sheath 110 in the bone tunnel 22. A clamp may be temporarily placed on the activation strand 160 to ensure that the suture construct 100 is anchored.

With the suture construct 100 anchored in the bone tunnel 22, the soft tissue 10 may be repaired and secured to the bone tissue 20 with direct visualization. The lengths 170, 171 may remain outside of the bone tunnel 22 and available for use in securing the soft tissue 10. The first length 170 may be loaded onto a suture passer (e.g., a Scorpion™ as produced by Arthrex, not shown) to be passed through a first portion of the soft tissue 10. The first length 170 may then be threaded through the loop 184 of the first shuttle 180. The second length 171 may be loaded onto the suture passer to be passed through a second portion of the soft tissue 10. The second length 171 may then be threaded through the loop 184 of the second shuttle 181. The clamp may be released from the activation strand 160, and each of the proximal portions 186 of the shuttles 180, 181 may be pulled to retract each of the length 170, 171 into the respective spliced portions 174 to form the knotless anchor. Each of the activation strand 160 and the shuttles 180, 181 may be independently tensionable to ensure that the soft tissue 10 is securely tensioned. The act of bunching the sheath 122 deforms the surrounding cancellous bone, securing the suture construct 100. The activation strand 160 may be cut at the level of the distal exit of the tibial tunnel. The activation strand 160 may or may not be left in place once cut. For example, in the embodiment of FIGS. 1-2, the activation strand 160 may be cut and one limb may be pulled to unthread the activation strand 160 from the sheath 122. Once the soft tissue 10 is secured to the bone tissue 20, the retraction strand 150 may be removed to complete the soft tissue repair.

FIGS. 7A-C illustrate a first exemplary embodiment of loading the suture construct 100A with the repair strand 170, 171 and/or the at least one shuttle 180, 181, as described with reference to FIGS. 1-4. As illustrated in FIG. 7A, the at least one repair strand 170, 171 may be a single strand having a first length 170 and a second length 171. The second length 171 may be passed into the first opening 126 and through the lumen 112 of the first end portion 120 of the lumen 112 along the longitudinal axis L of the tubular sheath 110. The second length 171 may extend or be spliced out of the tubular sheath 110 at a first transverse opening 114 of the central portion 124. As illustrated in FIG. 7A, the second length 171 may form a loop 118 at least partially outside of the tubular sheath 110, bending backwards, and back into the lumen 112 at a second transverse opening 115 of the central portion 124. The loop 118 may extend 360° or be a closed loop (when viewed laterally). As further illustrated, the second length 171 may be passed through the lumen 112 of the second end portion 122 along the longitudinal axis L, and out of the second opening 128. The transverse opening 114 and the transverse opening 115 may be spaced apart along and/or traverse to the longitudinal axis L of the tubular sheath 110 in the first embodiment. The loop 118 being spliced through the tubular sheath 110 may fix the repair strand 170, 171 along the longitudinal axis L of the suture construct 100.

As further illustrated in FIG. 7C, the first shuttle 180 may extend or be spliced through the transverse opening 116 of the central portion 174 of the tubular sheath 110, through the first end portion 120, and out of the tubular sheath 110 through the first opening 126. Similarly, the second shuttle 181 may extend or be spliced through the transverse opening 117 of the central portion 174 of the tubular sheath 110, through the second end portion 122, and out of the tubular sheath 110 through the second opening 128. Although illustrated in the reverse order, the first shuttle 180 may extend or be spliced into the first length 170 and the second shuttle 181 may extend or be spliced into the second length 171 before each of the repair strand constructs is threaded into the tubular sheath 110. The transverse openings 116, 117 may be longitudinally spaced apart from each other and from the transverse openings 114, 115 along the longitudinal axis. Alternatively, as illustrated in FIGS. 3 and 4, the at least one shuttle 180, 181 may extend or be spliced out of the repair strand 170, 171 outside of the lumen 112 of the tubular sheath 110.

Thus, as illustrated in FIG. 7C, the repair strand 170, 171 may be fixed inside of the tubular sheath 110 by being spliced through the tubular wall of the tubular sheath 110, specifically by being looped through the transverse openings 114, 115. The first length 170 may extend through the first opening 126 and the first end portion 120. Similarly, the second length 171 may extend through the second opening 128 and the second end portion 122. The first shuttle 180 may extend or be spliced into the tubular sheath 110, into the first length 170, extend through the first opening 126, and have the proximal portion 186 exposed to be manipulated by the user. Similarly, the second shuttle 181 may extend or be spliced into the tubular sheath 110, into the second length 171, extend through the second opening 128, and have the proximal portion 186 exposed to be manipulated by the user. The lengths 170, 171 may be pulled taught to cinch the loop extending through the transverse openings 114, 115. In some embodiments, the repair strand 170, 171 may be further fixed with an adhesive and/or a weld.

The activation strand 160 may then extend or be spliced into the tubular sheath 110. Each free tail of the activation strand 160 may be inserted through a respective opening 126, 128 into the lumen of the tubular sheath 110. Each tail may extend or be spliced through a respective transverse opening, as discussed herein. Each tail may extend or be spliced into the other in an opposed fashion to create a continuous loop locked by the two opposing splices. The retraction strand 150 may be secured to the tubular sheath 110, as discussed herein.

FIGS. 8A-D illustrate a second exemplary embodiment of loading the suture construct 100B with the repair strand 170, 171 and/or the at least one shuttle 180, 181, as described with reference to FIGS. 1-4. The second embodiment 100B may be similar to the first embodiment 100A (the disclosure of which is incorporated herein by reference). In the second embodiment, the repair strand 170, 171 may be fixed to the tubular sheath 110 by being looped around the longitudinal axis L and being tied into a knot 173 inside of the lumen 112. As illustrated in FIG. 8A, the first length 170 and the second length 171 may be positioned on opposite sides of the longitudinal axis L of the tubular sheath 110. As illustrated in FIG. 8B, the repair strand 170, 171 may be wrapped or looped around the longitudinal axis L of the tubular sheath 110 at the central portion 124. The first length 170 may extend or be spliced into the tubular sheath 110 at the transverse opening 114, and the second length 171 may extend or be spliced into the tubular sheath 110 at the transverse opening 115. The transverse opening 114 and the transverse opening 115 may be spaced apart transverse of the longitudinal axis L of the tubular sheath 110 in the second embodiment. The first length 170 and the second length 171 may be manipulated to tie the knot 173 in the lumen 112 of the tubular sheath 110 to secure the repair strand 170, 171 to the tubular sheath 110. Thus, the loop around the longitudinal axis L of the tubular sheath 110 and the knot 173 may fix the first length 170 and the second length 171 relative to the tubular sheath 110, preventing relative movement of the lengths 170, 171 along the longitudinal axis L.

As illustrated in FIG. 8C, the first shuttle 180 may extend or be spliced into the first length 170, and the second shuttle 181 may extend or be spliced into the second length 171, as discussed herein. Splicing the shuttles 180, 181 outside of the lumen 112 and/or after forming knot 173 may reduce complexity in the splicing and/or knot forming.

As illustrated in FIG. 8D, the first length 170 and the first shuttle 180 may extend or be spliced into the lumen 112 at the transverse opening 116, through the first end portion 120, and out the first opening 126. Similarly, the second length 171 and the second shuttle 181 may extend or be spliced into the lumen 112 at the transverse opening 117, through the second end portion 122, and out the second opening 128. The transverse openings 116, 117 may be longitudinally spaced apart from each other and from the transverse openings 114, 115 along the longitudinal axis. However, it is also contemplated that the shuttles 180, 181 may extend or be spliced similar to that of the first embodiment.

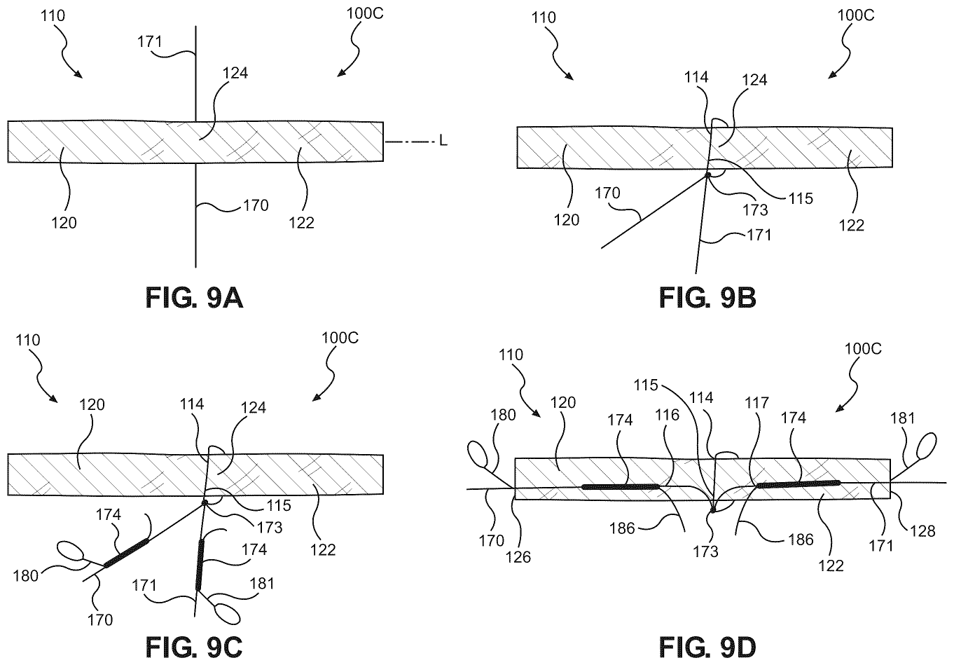

FIGS. 9A-D illustrate a third exemplary embodiment of loading the suture construct 100C with the repair strand 170, 171 and/or the at least one shuttle 180, 181, as described with reference to FIGS. 1-4. The third embodiment 100C may be similar to the first embodiment 100A and/or second embodiment 100B (the disclosure of which is incorporated herein by reference except when otherwise indicated). In the third embodiment, the knot 173 may be formed outside of the lumen 112. As illustrated in FIGS. 9A-B, the repair strand 170, 171 may be wrapped or looped around the longitudinal axis of the tubular sheath 110 similar to the second embodiment. As illustrated in FIG. 9B, the second length 171 may be spliced through the transverse opening 114 and out of the transverse opening 115 of the tubular sheath 110 to secure the repair strand 170, 171 to the tubular sheath 110, transverse to the longitudinal axis L. The first length 170 and the second length 171 may be manipulated to form the knot 173 outside of the lumen 112. The first length 170 may then be spliced into the tubular sheath 110 at the transverse opening 116, and the second length 171 may be spliced into the tubular sheath 110 at the transverse opening 117. The first length 170 of the repair strand may pass through the first end portion 120 and the first opening 126, and the second length 171 of the repair strand may pass through the second end portion 122 and the second opening 128. The shuttles 180, 181 may be spliced into the repair strand 170, 171 before or after threading the repair strand 170, 171 through the tubular sheath 110, as discussed with respect to at least one of the previous embodiments.

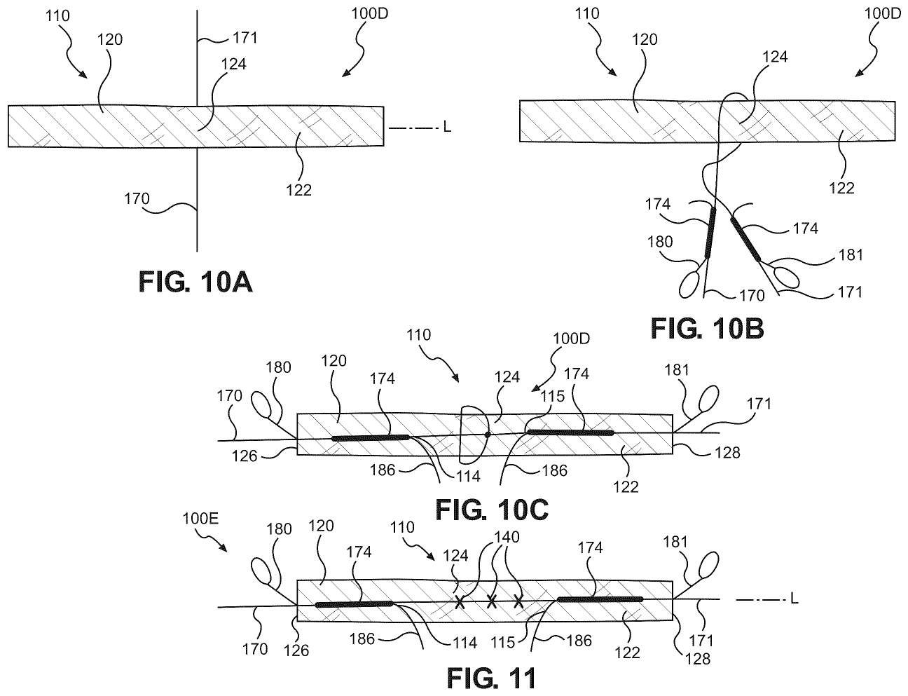

FIGS. 10A-C illustrate a fourth exemplary embodiment of loading the suture construct 100C with the repair strand 170, 171 and/or the at least one shuttle 180, 181, as described with reference to FIGS. 1-4. The first length 170 may extend or be spliced through the second length 171. Similarly, the second length 171 may extend or be spliced through the first length 170. The repair strands 170, 171 may be cinched down to a fixed loop around the sheath 110 to form a brummel splice.

FIG. 11 illustrates a fifth exemplary embodiment of loading the suture construct 100C with the repair strand 170, 171 and/or the at least one shuttle 180, 181, as described with reference to FIGS. 1-4. The fifth embodiment 100E may be similar to the at least one of the first through fourth embodiments 100A-D (the disclosure of which is incorporated herein by reference except when otherwise indicated). As illustrated, the repair strand 170, 171 may be threaded into and through the lumen 112 from the first opening 126 to second opening 128 along the longitudinal axis L. The repair strand 170, 171 may not be spliced through the wall of the tubular sheath 110 in the fifth embodiment. The suture construct 100C may further include one or more stitches 140 through the tubular wall of the tubular sheath 110 to fix the repair strand 170, 171 in the tubular sheath 110. The one or more stitches 140 may be threaded through the central portion 124 and compress the tubular wall to fix the repair strand 170, 171 longitudinally in place. The shuttles 180, 181 may be spliced into the repair strand 170, 171 before or after threading the repair strand 170, 171 through the tubular sheath 110, as discussed with respect to at least one of the previous embodiments.

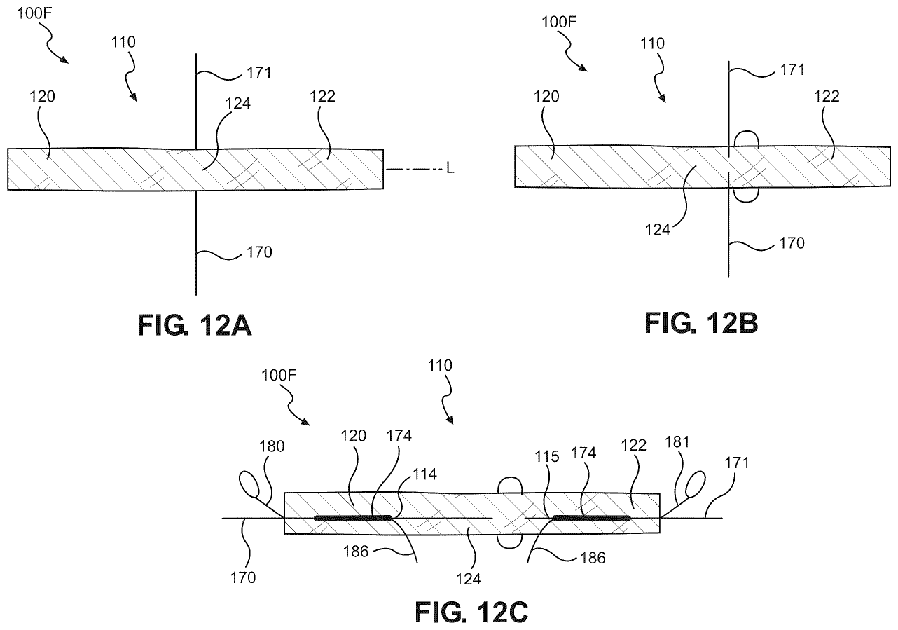

FIGS. 12A-C illustrate a sixth exemplary embodiment of loading the suture construct 100F with the repair strand 170, 171 and/or the at least one shuttle 180, 181, as described with reference to FIGS. 1-4. The sixth embodiment 100F may be similar to the at least one of the first through fifth embodiments 100A-E (the disclosure of which is incorporated herein by reference except when otherwise indicated). As illustrated, the repair strand 170, 171 may be spliced through the wall of the tubular sheath 110 at a plurality of openings, for example through the central portion 124. The openings of the splicing may be spaced apart along the longitudinal axis L and/or transverse to the longitudinal axis L. The first length 170 may then be spliced into the tubular sheath 110 at the transverse opening 114, and the second length 171 may be spliced into the tubular sheath 110 at the transverse opening 115. The first length 170 of the repair strand may pass through the first end portion 120 and the first opening 126, and the second length 171 of the repair strand may pass through the second end portion 122 and the second opening 128. The shuttles 180, 181 may be spliced into the repair strand 170, 171 before or after threading the repair strand 170, 171 through the tubular sheath 110, as discussed with respect to at least one of the previous embodiments.

FIGS. 13A-C illustrate a seventh exemplary embodiment of loading the suture construct 100G with the repair strand 170, 171 and/or the at least one shuttle 180, 181, as described with reference to FIGS. 1-4. The seventh embodiment may be similar to the at least one of the first through sixth embodiments (the disclosure of which is incorporated herein by reference except when otherwise indicated). As illustrated, the repair strand 170, 171 may be secured to the tubular sheath 110 through a luggage tag attachment. The repair strand 170, 171 may be folded (e.g., in half) to form a loop 176 on a first side of the tubular sheath 110. The first length 170 and the second length 171 of the repair strand may then be passed on a second side of the tubular sheath 110 and through the loop 176. The first length 170 and the second length 171 may be passed around the second side of the tubular sheath 110 and/or be spliced into the tubular wall at one or more locations along the second side. Thus, the repair strand 170, 171 may be looped at least partially around the tubular sheath 110 and through the loop 176 to form one or more loops 119. The first length 170 may then be spliced into the tubular sheath 110 at the transverse opening 114, and the second length 171 may be spliced into the tubular sheath 110 at the transverse opening 115. The first length 170 of the repair strand may pass through the first end portion 120 and the first opening 126, and the second length 171 of the repair strand may pass through the second end portion 122 and the second opening 128. The shuttles 180, 181 may be spliced into the repair strand 170, 171 before or after threading the repair strand 170, 171 through the tubular sheath 110, as discussed with respect to at least one of the previous embodiments.

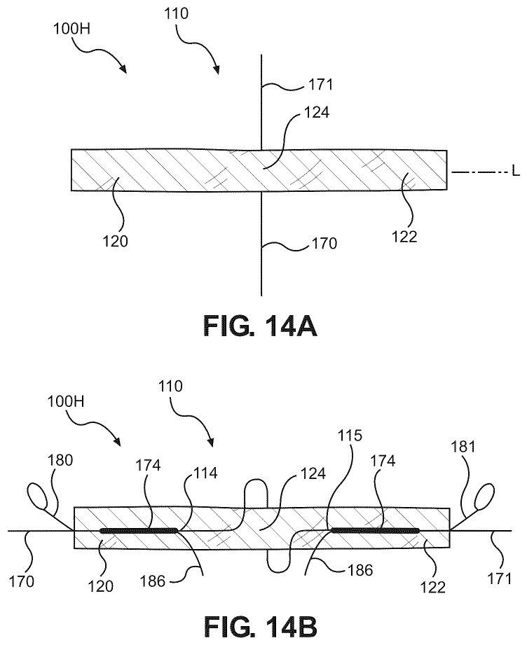

FIGS. 14A and 14B illustrate an eighth exemplary embodiment of loading the suture construct 100H with the repair strand 170, 171 and/or the at least one shuttle 180, 181, as described with reference to FIGS. 1-4. As illustrated, the repair strand 170, 171 may be spliced around the tubular sheath 110 at one or more locations along the circumference of the wall. After the initial splice of FIG. 11A, the repair strand 170, 171 may re-enter the sheath 110 on opposite sides. The first length 170 may be spliced into the tubular sheath 110 at the transverse opening 114, and the second length 171 may be spliced into the tubular sheath 110 at the transverse opening 115. The first length 170 of the repair strand may pass through the first end portion 120 and the first opening 126, and the second length 171 of the repair strand may pass through the second end portion 122 and the second opening 128. The repair strand 170, 171 may be fixed to the sheath 110 via frictional interaction, instead of relying on mechanical cinching, fixed loops, or direct fixation. The shuttles 180, 181 may be spliced into the repair strand 170, 171 before or after threading the repair strand 170, 171 through the tubular sheath 110, as discussed with respect to at least one of the previous embodiments.

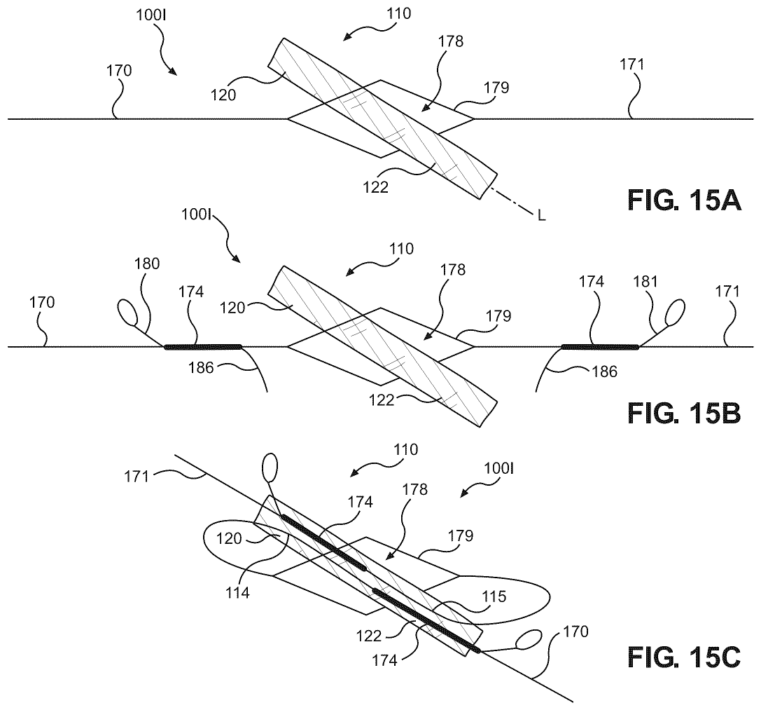

FIGS. 15A-C illustrate a ninth exemplary embodiment of loading the suture construct 100H, as described with reference to FIGS. 1-4. As illustrated, the repair strand 170, 171 may have a central opening 178 through which the tubular sheath 110 is threaded. The central opening 178 may be formed by a portion 179 that may be collapsed to retain the tubular sheath 110 therebetween. For example, the collapsible portion 179 may be formed by a scissor trap of a plurality of substantially straight segments. The first length 170 may then be spliced into the tubular sheath 110 at the transverse opening 114, and the second length 171 may be spliced into the tubular sheath 110 at the transverse opening 115. The first length 170 of the repair strand may pass through the first end portion 120 and the first opening 126, and the second length 171 of the repair strand may pass through the second end portion 122 and the second opening 128. The shuttles 180, 181 may be spliced into the repair strand 170, 171 before or after threading the repair strand 170, 171 through the tubular sheath 110, as discussed with respect to at least one of the previous embodiments.

It will also be appreciated by those skilled in the art that modifications can be made to the example embodiments described herein without departing from the invention. Structural features of systems and apparatuses described herein can be replaced with functionally equivalent parts or omitted entirely. Moreover, it will be appreciated that features from the embodiments can be combined with each other without departing from the disclosure.

What is claimed is:

1. A suture construct comprising:
a tubular sheath having a first end portion with a first opening, a second end portion with a second opening, and a lumen extending between the first opening and the second opening;
a repair strand in the lumen and having a length extending through the first end portion, wherein the repair strand is configured to engage a soft tissue;
a retraction strand configured to retract the tubular sheath; and
an activation strand in the lumen and configured to compress and anchor the tubular sheath in a bone tunnel, wherein the activation strand extends through the first opening and the second opening to tether the first end portion and the second end portion.

2. The suture construct of claim 1, wherein the activation strand is a closed loop.

3. The suture construct of claim 1, wherein the retraction strand comprises a loop extending around and/or through the tubular sheath.

4. The suture construct of claim 1, wherein the repair strand has a second length extending through the second end portion.

5. The suture construct of claim 1, wherein the repair strand is fixed to a portion of the tubular sheath.

6. The suture construct of claim 5, wherein the repair strand is fixed at a central portion between the first opening and the second opening.

7. The suture construct of claim 1, wherein the repair strand is spliced through the tubular sheath.

8. The suture construct of claim 7, wherein the repair strand is spliced at a first transverse opening and a second transverse opening of the tubular sheath.

9. The suture construct of claim 8, wherein the first transverse opening and the second transverse opening are spaced apart along a longitudinal axis the tubular sheath.

10. The suture construct of claim 9, wherein the repair strand forms a loop extending through the first transverse opening and the second transverse opening.

11. The suture construct of claim 8, wherein the first transverse opening and the second transverse opening are spaced apart transverse to a longitudinal axis of the tubular sheath.

12. The suture construct of claim 1, wherein the repair strand is looped at least partially around the tubular sheath.

13. The suture construct of claim 1, wherein the repair strand forms a knot inside or outside of the tubular sheath.

14. The suture construct of claim 13, wherein the repair strand forms the knot inside of the tubular sheath.

15. The suture construct of claim 1, wherein the repair strand is fixed to the tubular sheath with one or more stitches.

16. The suture construct of claim 1, wherein the repair strand includes a loop, the repair strand extends at least partially around the tubular sheath and through the loop, and the repair strand is spliced into the tubular sheath.

17. The suture construct of claim 1, wherein the repair strand has an opening, and the tubular sheath is passed through the opening.

18. The suture construct of claim 1, further comprising a shuttle associated with the repair strand.

19. The suture construct of claim 18, wherein the shuttle is spliced through a portion of the repair strand.

20. The suture construct of claim 19, wherein the portion of the repair strand is disposed entirely within the tubular sheath.

21. The suture construct of claim 19, wherein the portion of the repair strand is at least partially outside of the tubular sheath.

22. A method of forming a suture construct from a tubular sheath having a lumen, a first end portion, and a second end portion, the method comprising:
inserting a repair strand into the lumen of the tubular sheath, such that a length of the repair strand extends through the first end portion;
extending the repair strand through a first transverse opening and a second transverse opening of the tubular sheath;
inserting an activation strand into the tubular sheath; and
securing a retraction strand to the tubular sheath.

23. The method of claim 22, wherein the activation strand extends through a first opening of the first end portion and a second opening of the second end portion to tether the first end portion and the second end portion.

24. The method of claim 22, wherein the first transverse opening and the second transverse opening are spaced apart along a longitudinal axis of the tubular sheath.

25. The method of claim 22, wherein the first transverse opening and the second transverse opening are spaced apart transverse to a longitudinal axis of the tubular sheath.

26. The method of claim 22, further comprising looping the repair strand at least partially around the tubular sheath.

27. The method of claim 22, further comprising forming a knot with the repair strand inside or outside of the tubular sheath.
